(19) United States
(12) Patent Application Publication
(10) Pub. No.: US 2024/0269210 A1
(43) Pub. Date: Aug. 15, 2024

(54) METHOD FOR ENHANCING BIOLOGICAL ENERGY WITH EXTRACT

(71) Applicant: TAIWAN MITOCHONDRION APPLIED TECHNOLOGY CO., LTD., Zhubei City (TW)

(72) Inventors: Shun-Chieh Yang, Zhubei City (TW); Han-Chung CHENG, Zhubei City (TW)

(21) Appl. No.: 18/436,561

(22) Filed: Feb. 8, 2024

(30) Foreign Application Priority Data
Feb. 10, 2023 (TW) 112104722

(51) Int. Cl. A61K 36/232; A61K 31/12; A61K 31/366; A61P 43/00

(52) U.S. Cl. CPC A61K 36/232; A61K 31/12; A61K 31/366; A61P 43/00

(57) ABSTRACT

A method of enhancing biological energy with an extract is disclosed. The activity-enhanced mitochondria can maintain their function and activity when encountering stress to ensure normal work of cells without being affected by external or internal stress.

### CROSS-REFERENCE TO RELATED APPLICATIONS

This non-provisional application claims priority under 35 U.S.C. § 119(a) on Patent Application No. 112104722 filed in Taiwan (R.O.C.) on Feb. 10, 2023, the entire contents of which are hereby incorporated by reference.

### BACKGROUND

### 1. Technical Field

The present disclosure relates to a method for enhancing biological energy with an extract, particularly to a method for enhancing biological energy with an ashitaba extract.

### 2. Related Art

Mitochondria (called “mitochondrion” in singular form) are places where oxidative phosphorylation (OXPHOS) and adenosine triphosphate (ATP) synthesis occur. Since ATP is used as a source of energy in a cell, the mitochondria are described as the powerhouse of the cell. In addition to generating the energy required by the cell, the mitochondria also participate in cell differentiation, cell signaling, apoptosis of the cell, and so on, and the mitochondria have the ability to control the cell-division cycle.

However, some of the side products generated in the oxidative phosphorylation are harmful to mitochondria, such as reactive oxygen species (ROS) including superoxide anion (O_{2·}⁻), perhydroxyl radical (HO_{2·}), hydrogen peroxide (H₂O₂), and the like. ROS has a high bioactivity and may easily cause oxidative damage to cells or mitochondria. The damaged mitochondria may have adverse effects on cell energy supply and cell growth. For a long time, the severely damaged mitochondria may release cytochrome c (Cyt c), caspase, procaspase-2, procaspase-3, procaspase-8, procaspase-9, and the like, these may trigger the collapse of mitochondria, and the severely damaged mitochondria may also release apoptosis-related signaling factors, thereby triggering the apoptosis. Therefore, how to enhance the ability of mitochondria to cope with stress, protect and repair mitochondria to maintain their functions, and reduce the collapse of mitochondria has become an important issue.

### SUMMARY

According to one or more embodiments of the present disclosure, there provides a method for enhancing biological energy with an extract, comprising administrating an effective amount of the extract to a subject, wherein the extract is an ashitaba extract, and the ashitaba extract is extracted from Angelica keiskei.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 shows the result of the cytotoxicity test for the ashitaba extract with different concentrations;

FIG. 2 shows the oxygen consumption of the mitochondria for overcoming proton leakage;

FIG. 3 shows the oxygen consumption of the mitochondria for ATP production;

FIG. 4 shows the oxygen consumption of the mitochondria for spare respiration;

FIG. 5 shows the oxygen consumption of the mitochondria for maximal respiration; and

FIG. 6 shows the ATP coupling efficiency of the mitochondria.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

The ashitaba extract in the embodiments of the present disclosure is derived from Angelica keiskei. Angelica keiskei, also known as ashitaba, literally “tomorrow's leaf”, is originally from Hachijojima, Japan, and can thrive in temperate regions at low altitudes and in subtropical regions with an altitude of 500 to 2000 meters above sea level. The ashitaba is known for its strong vitality, earning its name from the characteristic of “leaves are harvested today, and new buds grow tomorrow.” The ashitaba has abundant chlorophyll, vitamins, dietary fiber, proteins, amino acids, and various minerals essential for the human body. It is reported that the ashitaba is beneficial to human health, with its stems and leaves used in health supplements, and its roots utilized as medicinal herbs or food additives.

The main bioactive components of the ashitaba include coumarins and chalcones. Coumarin compounds exhibit antioxidant, anticancer, antidepressant, and acetylcholinesterase inhibitory effects, while chalcone compounds exhibit antioxidant, anticancer, and glucosidase inhibitory effects. In the leaves of the ashitaba, the coumarin compounds mainly include xanthotoxin and laserpitin, while the chalcone compounds mainly include xanthoangelol and 4-hydroxyderricin, and the chalcone compounds in the leaves of the ashitaba exhibit xanthine oxidase inhibitory effects.

The extract of one embodiment of the present disclosure is an ashitaba extract, which may be obtained by cracking ashitaba into powder, soaking the powder in water at a ratio of 1 gram of powder: 25 milliliters of water at room temperature for 1 day, centrifuging the obtained solution, and freeze-drying the obtained supernatant. In detail, the leaves of the ashitaba are washed and dried, cracked into powder with pneumatic cracking (60 mesh), and then grading-selected, and the powder is inspected with a metal detector to prevent metal impurities from being introduced during the process. The inspected ashitaba powder is soaked in water at a ratio of 1 gram of powder: 25 milliliters of water at room temperature for 1 day, that is, extracted with water at normal temperature for 1 day. The obtained solution is centrifuged at 3000 g for 30 minutes, and the obtained supernatant after centrifugation is freeze-dried, thereby obtaining the powder of the ashitaba extract used in the embodiments of the present disclosure. The obtained powder is prepared into an aqueous solution, which is the ashitaba extract used in the embodiments of the present disclosure.

The ashitaba extract obtained by extraction as described above includes xanthotoxin, xanthoangelol, 4-hydroxyderricin, and laserpitin. The amount of xanthotoxin may be 0.95 mg/g to 1.05 mg/g, the amount of xanthoangelol may be 1.05 mg/g to 1.15 mg/g, the amount of 4-hydroxyderricin may be 0.55 mg/g to 0.65 mg/g, and the amount of laserpitin may be 0.75 mg/g to 0.85 mg/g.

In some embodiments of the present disclosure, biological energy of cells of an subject may be enhanced by providing the cells of the subject with the ashitaba extract with an effective amount of 250 μg/mL to 1000 μg/mL. The biological energy may be represented by the activity of mitochondria. More specifically, the spare respiratory capacity, the maximal respiratory capacity, and the ATP production of the mitochondria may be increased, the proton leakage of the mitochondria may be decreased, and the ATP coupling efficiency and Bioenergetic Health Index (BHI) of the mitochondria may be increased. In another embodiment, the effective amount of the ashitaba extract may be 250 μg/mL to 500 μg/mL. In the other embodiment, the effective amount of the ashitaba extract may be 500 μg/mL to 1000 μg/mL.

As a manner for providing cells of a subject with the ashitaba extract, for example, the ashitaba extract may be taken in by the subject by oral administration. When the ashitaba extract is provided by oral administration, the effective dose of the ashitaba extract may be from 2.703 g to 10.812 g. The effective dose in human is obtained according to a conversion equation. The conversion equation is: (effective dose in human)=(effective dose in cell experiment)×(body weight of mice)×(conversion coefficient)×(body weight of human). The conversion coefficient is obtained from the conversion coefficient table. For example, when the body weight of mice is 20 g and the body weight of human is 60 kg, the conversion coefficient is 9.01. In another embodiment, the effective dose of the ashitaba extract may be 2.703 g to 5.406 g. In the other embodiment, the effective dose of the ashitaba extract may be 5.406 g to 10.812 g.

To make the oral administration more convenient, the ashitaba extract may be made into a processed food, and the processed food may be provided in liquid form, solid form, powder form, granular form, paste form, or colloidal form. In some embodiments of the present disclosure, without being affected the effect and the purpose of the present disclosure, the processed food of the ashitaba extract may also include other ingredients or additives, such as a carrier, a diluent, an adjuvant, an excipient, or a flavor enhancer. The excipient may make the formulation convenient and practical, and the flavor enhancer may improve the flavor of the formulation.

For example, the excipient may be starch, such as wheat starch, rice starch, corn starch, potato starch, dextrin, cyclodextrin, and the like; crystalline cellulose; saccharide, such as lactose, glucose, sugar, reduced maltose, cerealose, oligofructose, galactooligosaccharide, and the like; or glycitol, such as sorbitol, erythritol, xylitol, lactitol, mannitol, and the like.

For example, the flavor enhancer may be fruit extract, such as longan extract, lychee extract, grapefruit extract, and the like; fruit juice, such as apple juice, orange juice, lemon juice, and the like; essence, such as peach essence, plum essence, yogurt essence, and the like; sweetener, such as acesulfame potassium, sucralose, erythritol, oligosaccharide, mannose, xylitol, isomerized sugar, and the like; acid flavoring, such as citric acid, malic acid, tartaric acid, gluconate, and the like; or tea ingredient, such as green tea, oolong tea, banaba tea, eucommia tea, tieguanyin tea, coix tea, jiaogulan tea, zizania latifolia tea, kelp tea, and the like.

Moreover, the ashitaba extract of the present disclosure may be made into a pharmaceutical composition or a non-pharmaceutical composition, such as health supplement. The ashitaba extract or the composition including the ashitaba extract may be encapsulated in a capsule for convenient oral administration. The ashitaba extract or the composition including the ashitaba extract may be encapsulated in a hard capsule in a dried powder form. Also, the ashitaba extract or the composition including the ashitaba extract may be encapsulated in a soft capsule in a liquid form, a suspension form, a paste form, a powder form, or a granular form.

The oil in the soft capsule for dissolving the ashitaba extract may be, for example, avocado oil, almond oil, flaxseed oil, fennel oil, Perilla frutescens oil, olive oil, olive squalene, sweet orange oil, orange roughy oil, sesame oil, garlic oil, cocoa butter, pumpkin seed oil, chamomile oil, carrot oil, cucumber oil, tallow fatty acid, kukui nut oil, lingonberry seed oil, in brown rice germ oil, rice bran oil, wheat germ oil, safflower oil, shea butter, liquid shea butter, perilla oil, soybean oil, evening primrose oil, camellia oil, corn oil, rapeseed oil, saw palmetto extract oil, coix oil, peach kernel oil, celery seed oil, castor oil, sunflower oil, grapeseed oil, borage oil, macadamia nut oil, meadowfoam oil, cottonseed oil, peanut oil, turtle oil, mink oil, egg yolk oil, fish oil, palm oil, palm-kernel oil, wood wax oil, coconut oil, long-chain/medium-chain/short-chain triglyceride, diglyceride, butter, lard, squalene, squalane and pristane and hydrides thereof.

In addition, several food additives approved for use, such as colorant, preservative, tackifier, binder, disintegrant, dispersant, stabilizer, gelatinizer, antioxidant, surfactant, preservative, and pH control agent, may be added to the processed food of the ashitaba extract according to relevant regulations and manufacturing requirements.

The following demonstrates the experiments for the ashitaba extract of the present disclosure enhancing the activity of mitochondria. The following experiments are conducted by using skeletal muscle cells (C2C12 cells). The cell culture is performed in DMEM with 10% fetal bovine serum (FBS). The cell subculture is described as follows. First, the skeletal muscle cells are cultured to a certain amount, and then the culture medium is removed. The skeletal muscle cells are rinsed with phosphate buffered saline (PBS) twice. Then, trypsin is added to react with the skeletal muscle cells at 37° C. for 5 minutes, and then the culture medium is added thereto to stop the reaction of trypsin. Then, the mixture is centrifuged at 300 g for 5 minutes to remove the supernatant, and the pellet containing the skeletal muscle cells is resuspended with the culture medium. Finally, the skeletal muscle cells are transferred to a 175T flask for subsequent experiments, and the cell count in the 175T flask is 1×10⁶ cells.

### Experiment 1: The Cytotoxicity Test for the Ashitaba Extract

First, the cytotoxicity test for the ashitaba extract is conducted. Alamar blue is a cell viability assay reagent. In the Alamar blue cell viability assay kit, resazurin is a redox indicator, which is a nontoxic, cell-permeable, weakly fluorescent, and deep blue dye. Upon entering living cells, resazurin is reduced to resorufin, a compound that is pink and highly fluorescent, due to the reducing environment in the living cells. The cell viability may be evaluated by detecting the absorbance or fluorescence of resorufin. The higher absorbance or fluorescence of resorufin indicates the higher cell viability. High viability means that the cells are healthy and have a high proliferation ability. When the cells have a high proliferation ability, the amount of the cells increases. Therefore, Alamar blue may be used as an indicator of cytotoxicity to reflect cell viability and cell proliferation.

The procedure for the cytotoxicity test of the ashitaba extract is described as follows. On the first day, the skeletal muscle cells are cultured in a 96-well plate with a total volume of 200 μL and 10000 cells per well for one day. On the second day, the ashitaba extract is added, and the concentrations of the ashitaba extract in each well are 50, 100, 200, 250, 500, and 1000 μg/mL. The skeletal muscle cells are incubated at 37° C. for one day. On the third day, the cytotoxicity test is conducted with Alamar blue. In detail, Alamar blue is prepared to a solution of 10 wt % in a dark environment, added to the 96-well plate with 100 μL per well, and incubated with the skeletal muscle cells at 37° C. for 3 to 4 hours. Then, the absorbance and fluorescence (OD530/590) are measured by ELISA reader, and the cell viability of the skeletal muscle cells after being treated with the ashitaba extract is obtained so as to represent the cytotoxicity of the ashitaba extract.

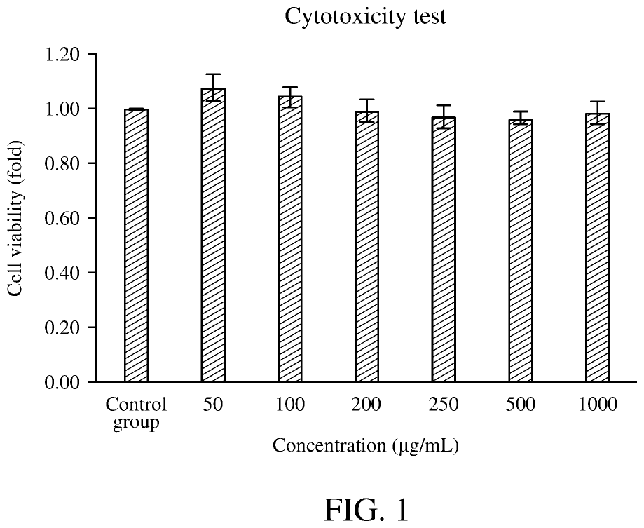

FIG. 1 shows the result of the cytotoxicity test for the ashitaba extract with different concentrations. The control group is the skeletal muscle cells not treated with the ashitaba extract (0 μg/mL), and the vertical axis is the fold of the cell viability relative to the control group.

As shown in FIG. 1, the ashitaba extract less than 1000 μg/mL has no adverse effect on the cell viability. This indicates that the ashitaba extract less than 1000 μg/mL has no cytotoxicity. Accordingly, 250, 500, and 1000 μg/mL of the ashitaba extract are selected as Examples 1 to 3 of the present disclosure for the subsequent experiments.

### Experiment 2: Enhancing the Activity of the Mitochondria with the Ashitaba Extract

Next, the experiment for enhancing the activity of the mitochondria with the ashitaba extract is conducted. In the experiment, tert-butyl hydroperoxide (t-BHP) is used as a substance that induces cellular oxidative stress damage and aging and inhibits the activity of the mitochondria.

The experimental procedure for enhancing the activity of the mitochondria with the ashitaba extract is described in detail as follows. On the first day, the skeletal muscle cells are cultured with the culture medium in a 24-well plate for Seahorse XF analysis with a total volume of 100 μL and 25000 cells per well for 4 hours, and then 150 μL of the culture medium is added and incubated for one day. On the second day, the ashitaba extract is added, and the concentrations of the ashitaba extract in each well are 250, 500, and 1000 μg/mL with a total volume of 250 μL in each well. The skeletal muscle cells are incubated with the ashitaba extract for one day. On the third day, the culture medium is replaced with fresh culture medium, 100 μM of t-BHP is added to each well and reacted with the skeletal muscle cells for 1 hour, and then the culture medium in each well is replaced with 675 μL of the medium, a DMEM medium without FBS, for measuring, and incubated in an incubator without CO₂ for 1 hour. Then, the oxygen consumption of the skeletal muscle cells in each well is measured by a Seahorse XF analyzer.

The principle and procedure of Seahorse XF analyzer are described as follows. First, the oxygen consumption for the basal respiration of the cells is measured. Then, a ATP synthesis inhibitor is added to inhibit the mitochondria from synthesizing ATP, and the reduction of the oxygen consumption is equal to the oxygen consumption for the ATP production. Then, an anti-coupler in a proper concentration, which causes no damage to the electron transport chain in the inner mitochondrial membrane, is added to evaluate the oxygen consumption for the maximal respiration of the mitochondria. Finally, an electron transport chain inhibitor is added to totally stop the respiration in the mitochondria, and the background is measured, which is equal to the oxygen consumption for the non-mitochondrial respiration. The oxygen consumption of the basal respiration of the mitochondria is equal to the oxygen consumption of the basal respiration of the cells minus the oxygen consumption of the non-mitochondrial respiration. The oxygen consumption for overcoming proton leakage of the mitochondria is equal to the oxygen consumption for the basal respiration of the mitochondria minus the oxygen consumption of the mitochondria for the ATP production. The oxygen consumption for the spare respiration of the mitochondria is equal to the oxygen consumption for the maximal respiration of the mitochondria minus the oxygen consumption for the basal respiration of the mitochondria. The ATP coupling efficiency is equal to the oxygen consumption of the mitochondria for the ATP production divided by the oxygen consumption for the basal respiration of the mitochondria.

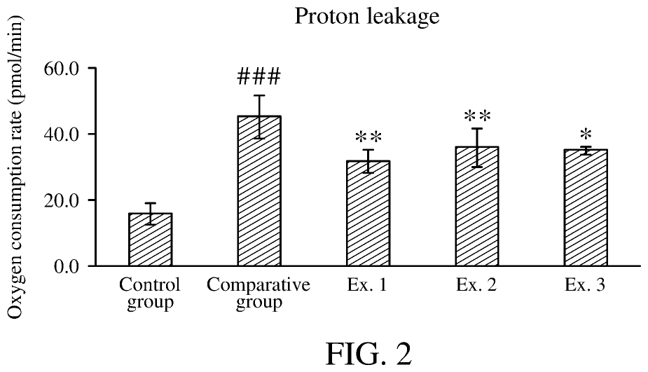

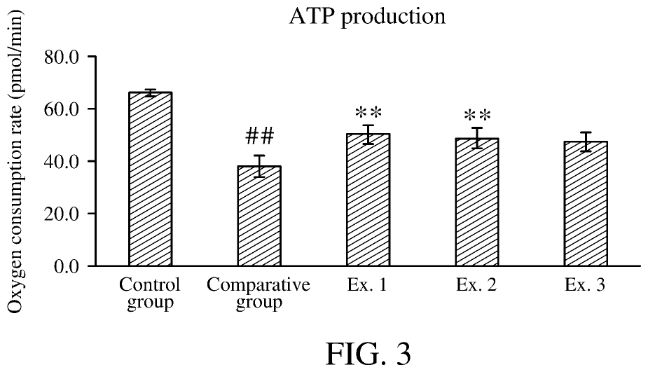

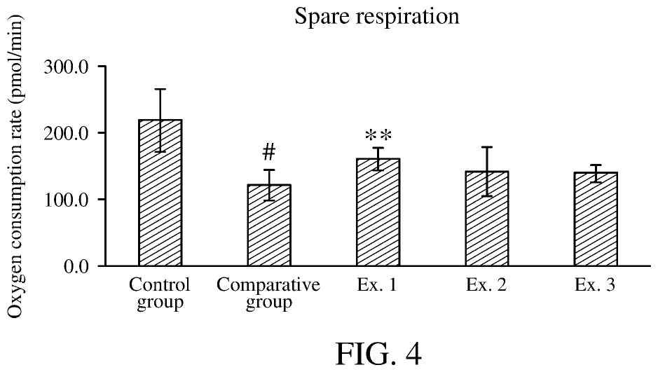

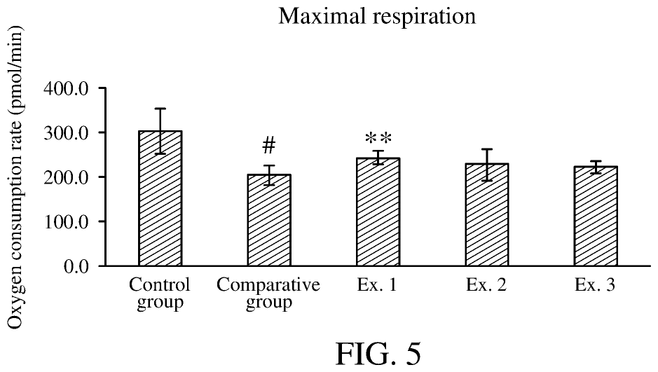

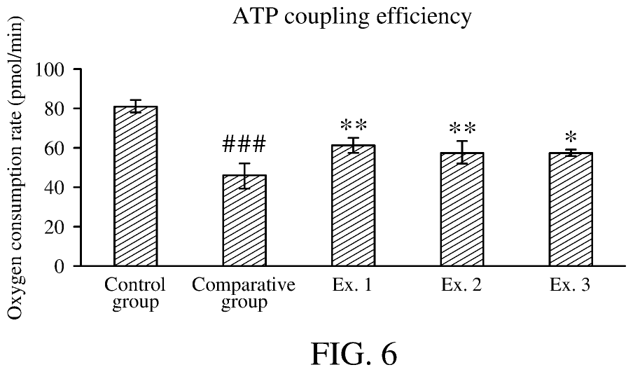

The results are shown in Table 1 and FIGS. 2 to 6. FIG. 2 shows the oxygen consumption of the mitochondria for overcoming proton leakage; FIG. 3 shows the oxygen consumption of the mitochondria for ATP production; FIG. 4 shows the oxygen consumption of the mitochondria for spare respiration; FIG. 5 shows the oxygen consumption of the mitochondria for maximal respiration; and FIG. 6 shows the ATP coupling efficiency of the mitochondria. The control group (Con.) is the skeletal muscle cells not treated with t-BHP and the ashitaba extract, the comparative group (Comp.) is the damaged skeletal muscle cells treated with t-BHP but not with the ashitaba extract, and the experimental groups (Example, Ex.) are the skeletal muscle cells treated with the ashitaba extracts of Examples 1 to 3, respectively, and then treated with t-BHP. In FIGS. 2 to 5, the vertical axis is the oxygen consumption in pmol per minute. In FIG. 6, the vertical axis is the ATP coupling efficiency in percentage (%). Symbols “*” and “**” mean there is a statistically significant difference between the experimental groups and the comparative group (*: P<0.05; **: P<0.01), and symbols “#”, “##”, and “###” mean there is a statistically significant difference between the comparative group and the control group (#: P<0.05; ##: P<0.01; ###: P<0.001).

TABLE 1

| | | | | | | | Non- | ATP |
|---|---|---|---|---|---|---|---|---|
| | Ashitaba | Basal | Proton | ATP | Maximal | Spare | mitochondrial | coupling |
| | extract | respiration | leakage | production | respiration | respiration | respiration | efficiency |
| Unit | μg/mL | pmole/min | | | | | | % |
| Con. | — | 81.37 ± | 15.64 ± | 65.73 ± | 300.47 ± | 219.10 ± | 18.63 ± | 80.87 ± |
| | | 3.67 | 3.16 | 1.36 | 49.77 | 46.52 | 3.67 | 3.19 |
| Comp. | — | 83.13 ± | 45.12 ± | 38.01 ± | 204.21 ± | 121.08 ± | 16.87 ± | 45.84 ± |
| | | 2.21 | 6.33 | 4.21 | 21.11 | 23.12 | 2.21 | 3.14 |
| Ex. 1 | 250 | 81.76 ± | 31.75 ± | 50.01 ± | 242.09 ± | 160.33 ± | 18.24 ± | 61.18 ± |
| | | 1.49 | 3.42 | 3.33 | 15.22 | 15.63 | 1.49 | 4.02 |
| Ex. 2 | 500 | 84.49 ± | 35.91 ± | 48.58 ± | 226.69 ± | 142.20 ± | 15.51 ± | 57.61 ± |
| | | 2.30 | 5.75 | 3.60 | 34.23 | 35.89 | 2.30 | 5.67 |
| Ex. 3 | 1000 | 82.04 ± | 34.97 ± | 47.07 ± | 220.72 ± | 138.67 ± | 17.96 ± | 57.33 ± |
| | | 4.10 | 1.15 | 3.47 | 13.61 | 11.79 | 4.10 | 1.64 |

As shown in FIG. 2, in terms of the oxygen consumption of the mitochondria for overcoming proton leakage, the comparative group is higher than the control group. This means that in the comparative group, the inner membrane of the mitochondria is damaged so that more oxygen is needed to overcome the proton leakage. In contrast, the experimental groups treated with the ashitaba extract of Examples 1 to 3 are less than the comparative group. This means that the activity of the mitochondria in the experimental groups is enhanced by the ashitaba extract, and also means that the ashitaba extract is able to protect and repair the mitochondria under oxidative stress so that the inner membrane of the mitochondria is less damaged.

As shown in FIG. 3, in terms of the oxygen consumption of the mitochondria for the ATP production, the comparative group is less than the control group. This means that in the comparative group, the ability to synthesize ATP of the mitochondria under oxidative stress is decreased and the energy generated by the mitochondria is also decreased. In contrast, the experimental groups treated with the ashitaba extract of Examples 1 to 3 are higher than the comparative group. This means that the activity of the mitochondria in the experimental groups is enhanced by the ashitaba extract, and the ability to synthesize ATP of the mitochondria under oxidative stress in the experimental groups is also increased so that the mitochondria can generate enough energy for the cells.

As shown in FIG. 4, in terms of the oxygen consumption of the mitochondria for the spare respiration, the comparative group is less than the control group. This means that in the comparative group, the spare respiration capacity of the mitochondria under oxidative stress is decreased. In contrast, the experimental groups treated with the ashitaba extract of Examples 1 to 3 are higher than the comparative group. This means that the activity of the mitochondria in the experimental groups is enhanced by the ashitaba extract, and the spare respiration capacity of the mitochondria under oxidative stress in the experimental groups is also increased. The increase of the spare respiration capacity of the mitochondria means that the mitochondria have an enhanced ability to cope with stress.

As shown in FIG. 5, in terms of the oxygen consumption of the mitochondria for the maximal respiration, the comparative group is less than the control group. This means that in the comparative group, the maximal respiration capacity of the mitochondria under oxidative stress is decreased. In contrast, the experimental groups treated with the ashitaba extract of Examples 1 to 3 are higher than the comparative group. This means that the activity of the mitochondria in the experimental groups is enhanced by the ashitaba extract, and the maximal respiration capacity of the mitochondria under oxidative stress in the experimental groups is also increased.

As shown in FIG. 6, in terms of the ATP coupling efficiency, the comparative group is less than the control group, and the experimental groups treated with the ashitaba extract of Examples 1 to 3 are higher than the comparative group. This means that the activity of the mitochondria in the experimental groups is enhanced by the ashitaba extract, and the ATP coupling efficiency of the mitochondria under oxidative stress in the experimental groups is also increased.

Bioenergetic health index (BHI) may be calculated by the oxygen consumption of the mitochondria based on the results analyzed by Seahorse XF analyzer. BHI is an index for evaluating the energy metabolism of the mitochondria calculated by the mitochondrial energy metabolism data as parameters. BHI=log{[(the oxygen consumption for ATP production)×(the oxygen consumption of the spare respiration)]/[(the oxygen consumption for overcoming proton leakage)×(the oxygen consumption of the non-mitochondrial respiration)]}. The higher BHI of cells means the better activity of the mitochondria in the cells, as well as the better ability to cope with stress. Therefore, BHI may also be used as an indicator to evaluate the health of the mitochondria and the cells.

The BHI calculated by the above results of the oxygen consumption is shown in Table 2. As shown in Table 2, compared to the comparative group, the BHI of the mitochondria treated with the ashitaba extract of Examples 1 to 3 in the experimental groups is increased by the ashitaba extract. This means that the health of the mitochondria and the skeletal muscle cells are also improved.

TABLE 2

| | Ashitaba extract | |
|---|---|---|
| | (μg/mL) | BHI |
| Con. | — | 1.70 ± 0.09 |
| Comp. | — | 0.78 ± 0.13 |
| Ex. 1 | 250 | 1.14 ± 0.11 |
| Ex. 2 | 500 | 1.09 ± 0.16 |
| Ex. 3 | 1000 | 1.02 ± 0.13 |

According to the results described above, it can be seen that the ashitaba extract less than 1000 μg/mL has no cytotoxicity. Also, the ashitaba extract can enhance the activity of the mitochondria. More specifically, the spare respiratory capacity, the maximal respiratory capacity, and the ATP production of the mitochondria may be increased, the proton leakage of the mitochondria may be decreased, the ATP coupling efficiency of the mitochondria may be increased, and the BHI of the mitochondria may be increased.

According to the embodiments of the present disclosure, there provides a method for enhancing the activity of mitochondria with an extract, wherein the extract is an ashitaba extract, and the ashitaba extract may decrease the proton leakage of the mitochondria, increase the spare respiratory capacity, the maximal respiratory capacity, and the ATP production of the mitochondria, increase the ATP coupling efficiency of the mitochondria, and increase bioenergetic health index (BHI). The activity-enhanced mitochondria can maintain their function and activity when encountering stress to ensure normal work of cells without being affected by external or internal stress. In addition, the ashitaba extract also has effects on inhibiting tumor growth, improving inflammation, obesity, diabetes, and hypertension, as well as exhibiting anti-ulcer, anti-aging properties, and lowering blood pressure, blood lipids, blood sugar, and cholesterol.

What is claimed is:

1. A method for enhancing biological energy with an extract, comprising administrating an effective amount of the extract to a subject, wherein the extract is an ashitaba extract, and the ashitaba extract is extracted from Angelica keiskei.

2. The method of claim 1, wherein the enhancement of biological energy comprises decreasing proton leakage of mitochondria of the subject.

3. The method of claim 1, wherein the enhancement of biological energy comprises increasing ATP production of mitochondria of the subject.

4. The method of claim 1, wherein the enhancement of biological energy comprises increasing spare respiratory capacity of mitochondria of the subject.

5. The method of claim 1, wherein the enhancement of biological energy comprises increasing maximal respiratory capacity of mitochondria of the subject.

6. The method of claim 1, wherein the enhancement of biological energy comprises increasing ATP coupling efficiency of mitochondria of the subject.

7. The method of claim 1, wherein the enhancement of biological energy comprises increasing Bioenergetic Health Index (BHI) of mitochondria of the subject.

8. The method of claim 1, wherein the ashitaba extract comprises xanthotoxin, xanthoangelol, 4-hydroxyderricin, and laserpitin.

9. The method of claim 1, wherein the ashitaba extract is obtained by cracking ashitaba into powder, soaking the powder in water at a ratio of 1 gram of powder:25 milliliters of water at room temperature for 1 day, centrifuging the obtained solution, and freeze-drying the obtained supernatant.

10. The method of claim 1, wherein the effective amount of the ashitaba extract is 250 μg/mL to 1000 μg/mL.

11. The method of claim 1, wherein the biological energy is biological energy of skeletal muscle cells of the subject.
